(19) United States
(12) Patent Application Publication
(10) Pub. No.: US 2026/0363546 A1
(43) Pub. Date: Nov. 19, 2026

(54) Portable Slit Lamp

(71) Applicants: Nicholas Sears, Houston, TX (US); Hilary A. Beaver, Bellaire, TX (US); Nicholas J. Panzo, Houston, TX (US); Muhammad Hamza Memon, Houston, TX (US); Rijul Nanda, Houston, TX (US)

(72) Inventors: Nicholas Sears, Houston, TX (US); Hilary A. Beaver, Bellaire, TX (US); Nicholas J. Panzo, Houston, TX (US); Muhammad Hamza Memon, Houston, TX (US); Rijul Nanda, Houston, TX (US)

(73) Assignees: The Texas A&M University System, College Station, TX (US); The Methodist Hospital dba Houston Methodist, Houston, TX (US)

(21) Appl. No.: 19/675,986

(22) Filed: May 13, 2026

Related U.S. Application Data
(60) Provisional application No. 63/804,822, filed on May 13, 2025.

(51) Int. Cl. A61B 3/15; A61B 3/00; A61B 3/117

(52) U.S. Cl. CPC A61B 3/152; A61B 3/005; A61B 3/0058; A61B 3/1173

(57) ABSTRACT

The present invention is directed to a portable device or portable slit lamp that has a slit lamp with a head strap to position the slit lamp over a patient's head for vision examinations of anterior structures in the eyes. The portable slit lamp has at least one motor to motorize the slit, a modifiable light source to align with the slit, at least one camera to capture images, a microcontroller and network connection, and a power supply. The portable slit lamp is used for vision examinations in remote areas where images obtained during the examination are transmitted to a healthcare provider.

### CROSS-REFERENCE TO RELATED APPLICATIONS

This patent application claims benefit of priority under 35 U.S.C. § 119(e) of provisional application U.S. Ser. No. 63/804,822, filed May 13, 2025, the entirety of which are hereby incorporated by reference.

### BACKGROUND OF THE INVENTION

### Field of the Invention

The present invention relates generally to the fields of ocular diagnostic devices and telemedicine. Specifically, the present invention relates to a portable slit lamp useful for dedicated vision examinations in areas with limited or no access to healthcare providers to conduct such examinations.

### Description of the Related Art

Conventional slit lamps are largely unchanged over the last century and do not meet the current needs of ophthalmology. Conventional slit lamp biomicroscopy provides critical stereoscopic visualization for anterior segment assessment but is confined to fixed equipment-based clinical settings. Existing portable and remote systems either require an on-site operator or lack portability, limiting accessibility and autonomous tele-ophthalmic use.

The current widely used table-mounted slit lamp necessitates the patient and physician being in close proximity to conduct the exam. This makes slit lamp exams challenging in scenarios where there are restrictions to proximity between the two parties, such as those in rural populations. Patients in rural populations encounter increased barriers to ophthalmology care which can delay diagnoses and lead to reduced disease management.

Additionally, the findings of slit lamp examinations cannot be stored in electronic medical records for clinical, financial and legal purposes due to a lack of mechanisms that enable image capture. The inability to save exam findings in a pictorial format introduces a greater degree of subjectivity in interpretation and collaboration and inefficient transfer of care. Other factors such as geographical limitations add more complexity to provide comprehensive eye care. In some cases, traditional slit lamps may not be suitable for screening certain patients, necessitating the use of handheld slit lamps with reduced magnification and a lower clinical impact.

Thus, the prior art is deficient in a versatile ocular diagnostic device that enables healthcare providers to conduct a slit lamp examination in remote and resource-limited regions. Particularly, the prior art is deficient in a portable slit lamp integrating a dual-camera stereoscopic imaging system configured to transmit images to a healthcare provider. The present invention fulfills this long-standing need and desire in the art.

### SUMMARY OF THE INVENTION

The present invention is directed to a portable device for an ophthalmic examination. The portable device has a headset comprising a slit lamp integrated therein with a head strap attached thereto, means for remote access to results of the ophthalmic examination and a power source.

The present invention is further directed to a method for performing a vision examination on a patient in need thereof. In this method, positioning the portable device as described herein is positioned over the patient's head such that at least one camera in the slit lamp align with at least one eye. Images of at least one eye are obtained via the at least one camera during the vision examination. The images are transmitted wirelessly to a healthcare provider.

The present invention is directed further to a portable slit lamp configured for examination of anterior structures of both eyes. The portable slit lamp has a headset comprising at least one internal motor, a slit motorized via one of the internal motors, a modifiable light source aligned with the slit, means for capturing images of the eye, and a microcontroller with at least one network connection. The portable slit lamp also has a power supply. In a related embodiment, the portable slit lamp further comprises at least one of means for moving the modifiable light source in X-Y-Z directions; an autofocus; or stereoscopic imaging.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1D are pictures of the portable slit lamp. FIG. 1A is a model of an exterior view of portable slit lamp 100. FIG. 1B is a picture showing how the portable slit lamp and head strap fits on the head. FIG. 1C is a picture of the interior view of the portable slit lamp with the head strap. FIG. 1D is a picture of the dual-camera stereoscopic imaging assembly.

FIGS. 2A-2B are images demonstrating that slits moving across the pairs of eyes are visible.

FIGS. 3A-3B are real-time video (FIG. 3A) and still image (FIG. 3B) production of the slit.

FIGS. 4A-4B are images taken by the slit lamp (FIG. 4A) and by a smart phone (FIG. 4B).

FIGS. 5A-5B show examples of focus adjustment using the IMX-477 camera module.

FIGS. 6A-6C are representative stereoscopic image pairs from mannequin-eye testing at 10° (FIG. 6A), 40° (FIG. 6B), and 100° (FIG. 6C) fields of view (FOV).

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the conditional language, such as, among others, “can”, “might”, “may”, “e.g.”, “for example”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

In one embodiment of the present invention, there is provided a portable device for an ophthalmic examination, comprising a headset comprising a slit lamp integrated therein with a head strap attached thereto; means for remote access to results of the ophthalmic examination; and a power source.

In this embodiment, the head strap may be adjustable. Also, in this embodiment, the slit lamp may comprise a motorized light system with a slit; at least one camera module; and means for ambient lighting. In one aspect of this embodiment, the at least one camera module may comprise at least one camera for still images and at least one camera for live video imaging. In another aspect of this embodiment, the at least one camera module may be a dual-camera stereoscopic imaging assembly.

Further to this embodiment and aspects thereof the slit lamp may comprise means for X-Y-Z movement of the motorized light system; an autofocus; or stereoscopic imaging; or a combination thereof. In addition, the motorized light system with a slit may comprise a pair of slit LEDs. Furthermore, the ambient lighting means comprises an ambient LED.

Also in this embodiment and aspects thereof, the remote access means may comprise a microcontroller and at least one network connection. In addition, the portable device may be configured to examine anterior segments of both eyes. Particularly, the anterior segments may be segments of a cornea, of an iris or of a lens or a combination thereof.

In another embodiment of the present invention, there is provided a method for performing a vision examination on a patient in need thereof, comprising positioning the portable device as described supra over the patient's head such that at least one camera in the slit lamp align with at least one eye; obtaining images of at least one eye via the at least one camera during the vision examination; and transmitting wirelessly the images to a healthcare provider.

In an aspect of this embodiment, the obtaining step may comprise obtaining images of both eyes via cameras in a dual-camera stereoscopic imaging assembly. In another aspect of this embodiment, the patient may be remote from the healthcare provider, where the transmitting step may comprise transmitting wirelessly the images during a telemedicine consultation.

In yet another embodiment of the present invention, there is provided a portable slit lamp configured for examination of anterior structures of both eyes, comprising a headset comprising at least one internal motor; a slit motorized via one of the internal motors; a modifiable light source aligned with the slit; means for capturing images of the eye; a microcontroller with at least one network connection; and a power supply. Further to this embodiment, the headset comprises at least one of means for moving the modifiable light source in X-Y-Z directions; an autofocus; or stereoscopic imaging.

In one aspect of both embodiments the capturing means may comprise at least one camera for still images and at least one camera for live video. In another aspect, the capturing means may comprise a dual-camera stereoscopic imaging assembly.

In both embodiments and aspects thereof, the modifiable light source may comprise a pair of slit LEDs and an ambient LED. Also, the anterior structures of the eye are a cornea, an iris and a lens.

The present invention is directed to a headset-based portable slit lamp with a dual-camera stereoscopic imaging system integrated therein configured to capture both still images and live video. The device enables capture of high-quality images of each eye, along with live video that utilizes a narrow beam of light to visualize a magnified view of the anterior structures of the eye, including the cornea, iris, and lens. This feature not only enhances clinical workflows by allowing ophthalmologists to thoroughly review examination results via telemedicine or other wireless data transfer, but also facilitates consultations with other specialists, providing a more comprehensive approach to patient care. It is contemplated that the topography of the cornea may be evaluated and/or the posterior structures, for example, the retina and the optic nerve also may be visualized with adjustments to the slit lamp.

In addition, the device enables the capture of live video and detailed images enabling real-time demonstrations during patient exams, which can be used to educate residents, medical students, and other healthcare providers. Furthermore, with its live video capability, the device is particularly well-suited for use in remote settings where an ophthalmologist may not be physically present. The ability to perform telemedicine consultations using the device significantly reduces the time required for ophthalmologists to travel to hospitals or clinics, enabling rapid assessment of ocular conditions. This feature not only expands access to specialized care in underserved or rural or remote areas but also allows for quicker decision-making and potential treatment plans without the need to bring in outside ophthalmologists.

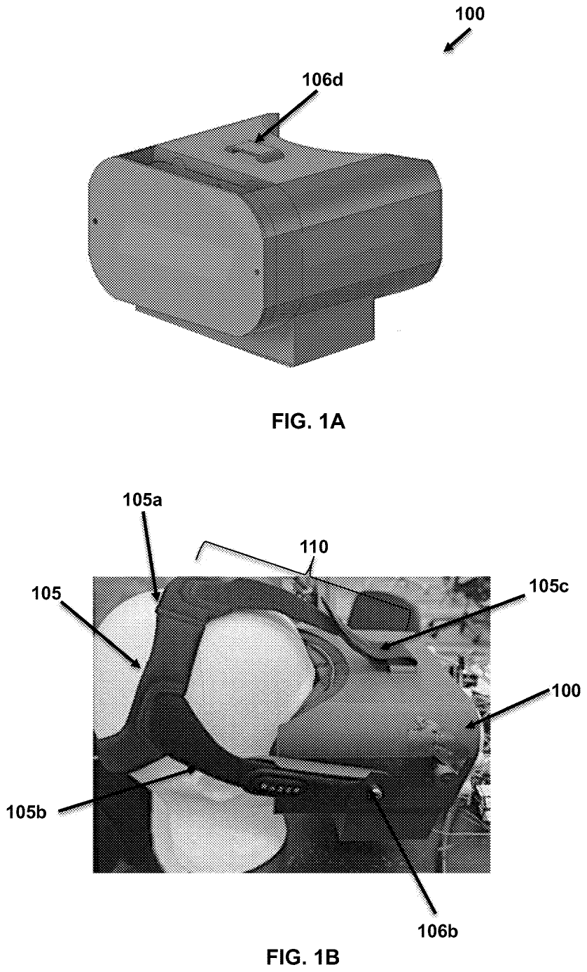

Particular embodiments of the present invention are better illustrated with reference to FIGS. 1A-1D, however, such reference is not meant to limit the present invention in any fashion. The embodiments and variations described in detail herein are to be interpreted by the appended claims and equivalents thereof.

FIG. 1A is a perspective view of the exterior of the portable slit lamp 100. A bracket 106b is secured to the top of the portable slit lamp through which a securing strap 105b is threadable (see FIG. 1B).

FIG. 1B shows the portable slit lamp with the head strap 105 attached thereto to make a wearable headset 110. The head strap has a circular portion 105a that encircles the back of the head with three securing straps 105b,c,d extending therefrom toward the mannequin's face and attached to the portable slit lamp. Securing strap 105d is not shown, but is attached to the right side of the portable slit lamp at 106d (see FIG. 1C) in a mirror image of securing strap 105c attached to the left side of the slit lamp at 106c. Securing strap 105b is adjustable to hold the portable slit lamp to the face. This strap threads through the bracket 106b such that end of the strap loops back onto itself and is secured via, for example, VELCRO. The headset is fitted onto a mannequin head to demonstrate positioning thereof and wearability.

FIG. 1C is a rear interior view of the headset 110 showing the padded liner 120 that creates a barrier between a patient's face and the headset, the head strap 105 and the space 130 within the portable slit lamp containing the moving components, lights or LEDS and cameras (see FIG. 1D) represented at 135. Securing strap 105d on the head strap is shown secured to the portable slit lamp at 106d.

FIG. 1D shows the dual-camera stereoscopic imaging assembly 140. The assembly has two movable cameras 140a,b with an adjustable horizontal baseline and ribbon cables 142a,b connected thereto which are designed to fit into the space 130 in the portable slit lamp. The cameras have an adjustable horizontal baseline so that they may be adjustable to align with a patient's eyes and to adjust focus.

The following examples are given for the purpose of illustrating various embodiments of the invention and are not meant to limit the present invention in any fashion.

### Example 1

### Experimental Design of the Portable Slit Lamp: Materials

The experimental design involved iterative design of the headset, iterative design of the motor code and hardware and continued improvement of the focus of the cameras and associated hardware.

Headset: The headset design began with an initial 3D print to determine feasibility. The successive prototypes were based on testing on overall size, fit and play of motors/lights/cameras, as well as a secondary endpoint of comfort. The design was completed in Fusion360 and printed on PLA. The iterative design to include the rail, followed by the pivot, then increased horizontal size to accommodate cameras, an Arduino, and a Raspberry Pi.

Motors: Stepper motors were utilized in the design. The parameters measured during experimentation were ability of motors to step, number of steps, heat dissipation, size, and sound. The materials utilized were Nema8 8HS11-0204S Bipolar motors, CNC shield, Arduino UNO, joysticks, and buttons. The protocols followed were software design principles of iterative software design and implementation, as well as the Nema8 standard for small motors.

LEDs: 3V/3W LEDs are utilized that are the standard for slit lamps. The experimental setup involved utilizing power/ground connections on an arduino to determine the brightness of the LEDs. There were no specific protocols utilized for this portion of the experimentation. A soldered connection for the LEDs was then developed and tested utilizing a multimeter.

Camera: 12.3 MP Arducam cameras attached to a Raspberry Pi are utilized. The cameras are tested based on their ability to focus (manually adjusted). The materials are the camera modules (off the shelf), Raspberry Pi, ribbon cables for the connection, a monitor and output for external display, mouse, keyboard, and software design of a python program to capture images from the camera.

X-Y-Z Movement: Integrating X-Y-Z movement into the device is essential for making it comparable to existing portable slit lamps (PSLs) and traditional slit lamps. This feature enables providers to adjust the light beam both horizontally and vertically, allowing for precise examination of different sections of the eye. This capability is particularly important for investigating various ocular conditions, such as detecting foreign bodies, evaluating corneal health, or assessing the anterior segment of the eye in detail. By incorporating X-Y movement, this tool meets the same level of adaptability and precision as current slit lamp technology.

Autofocus: Adding autofocus functionality sets the device apart from existing PSLs and traditional slit lamps, which generally lack this feature. Autofocus will automatically adjust the device's focus based on the distance of the eye's structures, eliminating the need for manual adjustments. This streamlines the examination process, reduce the overall time spent on each exam, and enhance the efficiency of clinical practice. By removing manual focus adjustments, practitioners can concentrate more on patient care and diagnostic interpretation, improving both workflow and patient experience.

Stereoscopic Imaging: Implementing dual cameras to provide stereoscopic imaging is another critical enhancement. This feature enables depth perception and a three-dimensional view of the eye's structures, which is a key capability of existing PSLs and traditional slit lamps. Stereoscopic imaging enables more accurate assessments of the anterior segment and facilitate better evaluation of structural changes and anomalies. By incorporating this feature, the device offers comprehensive imaging capabilities that match or exceed the current standards in ophthalmic diagnostics.

### Experimental Design of the Portable Slit Lamp: Methodology

Data collection for the iterative design process was carried out using a variety of tools. Fusion360 was used for designing the headset, while Arduino controlled the motors and LEDs, and Raspberry Pi managed the cameras. An online simulation tool helped with the initial lens design, enabling determination of the optimal distance between the lenses. However, the lenses were not included in the final design,

The headset was iterated upon to address user needs such as being lightweight and comfortable, while also meeting the size requirements for housing motors, cameras, LEDs, and electrical wiring. Each version of the design was 3D printed, including the final prototype. The initial prototype included two holes for the cameras and a slit at the bottom for wire management. Further iterations incorporated additional space for motor wiring and a slit for the ribbon cable of the camera, improving both functionality and ease of assembly.

The motors were tested in an iterative manner through software development. The motors purchased are of the NEMA8 standard. Motors were wired to fit a CNC shield atop an Arduino Uno. Testing then began utilizing the Arduino Uno stepper motor basic program. Following this, a new program was written for custom utilization for the design: the motors were to move smoothly to allow for analysis across the anterior surface of the eye with a 3D printed slit and LED on top. The primary variable adjusted to facilitate smooth movement was delay, as this controls the speed. The motors are controlled by joysticks connected to the CNC shield. Testing for the joysticks was the same as described for the motors, as the joysticks and the software control the speed of the motors.

The LEDs are powered directly from the Arduino Uno. The LEDs for the slit are powered via 5V, and the ambient light is powered via 3.3V. The ambient light may also be powered by the Raspberry Pi for simplification of the LED circuit. No significant testing was required for these given the off-the-shelf nature of the hardware.

The cameras are controlled by the Raspberry Pi. Testing involved ensuring that the hardware connection to the Raspberry Pi was stable, and that the Raspberry Pi software was capable of reading camera input. Ribbon cables provide the power and I/O connection from the camera to the Raspberry Pi. The cameras were then placed into the headset, and focused on the anterior surface of the eye, requiring manual adjustment through iterations of the design of the headset. Following this, a serial communication protocol was designed to allow for automatic control of the camera. That is, upon button press, the light scans the eye, and the cameras capture and store images automatically as the light moves.

The buttons were soldered onto a soldered breadboard. Signal and power are provided from the CNC shield. Two buttons control either motor (one per motor), with one reset button. Testing involved ensuring that the buttons move their respective motor, testing the hardware connection through a simple button press program, and ensuring that the button press sends a signal to the Raspberry Pi (via serial communication from the Arduino Uno) to capture and store images. The final prototype functions through a joystick mechanism.

### Example 2

### Results of Experimental Design

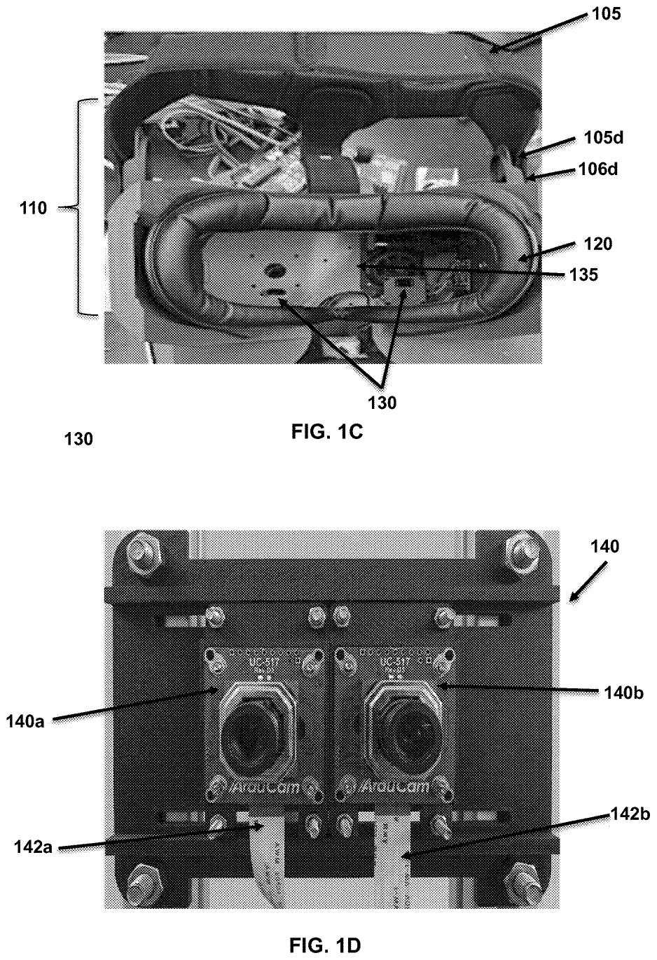

The final design is a headset form-factor slit lamp (FIGS. 1A-1C) capable of scanning a slit LED across both eyes with real-time image capture (FIG. 1D). This device features a single-button control that automates slit motion, image capture, and visualization. Its 12.3 MP camera delivers high-resolution imagery with full anterior segment visualization and superior clarity and focus. The images achieve a 133% absolute pixel improvement over traditional modalities used in low resource settings (FIGS. 2A-2B).

Particulars of the slit lamp are a:
1. Integrated design: mechanical, hardware, and software systems;
2. Ergonomic goggle-based model for extended use;
3. 3D-printed housing reinforced with metal fasteners;
4. LED slit illumination system with 3D-printed optics housing;
5. Stepper motors enable controlled slit movement across the anterior eye; and
6. Arduino microcontroller controls slit movement, while a Raspberry Pi (Python-based) manages image capture, processing, and display. Table 1 identifies specific features of the slit lamp.

TABLE 1
Features of the slit lamp

| Feature | Details |
|---|---|
| X, Y and Z-axis movement | X-axis: 100 mm |
| | Z-axis: 90 mm |
| | Y-axis: 30 mm |
| Illuminator movement | 45° to −45° |
| Illuminator type | Spot to slit |
| Scope output | Output to external display |

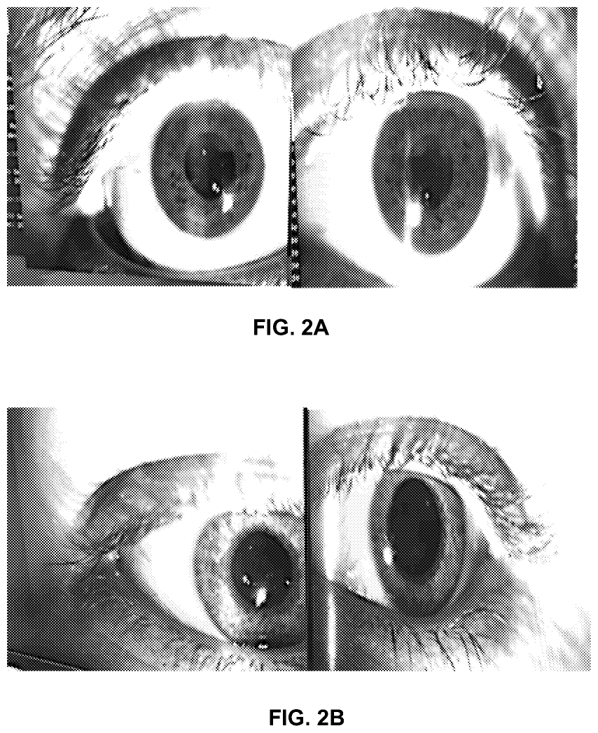

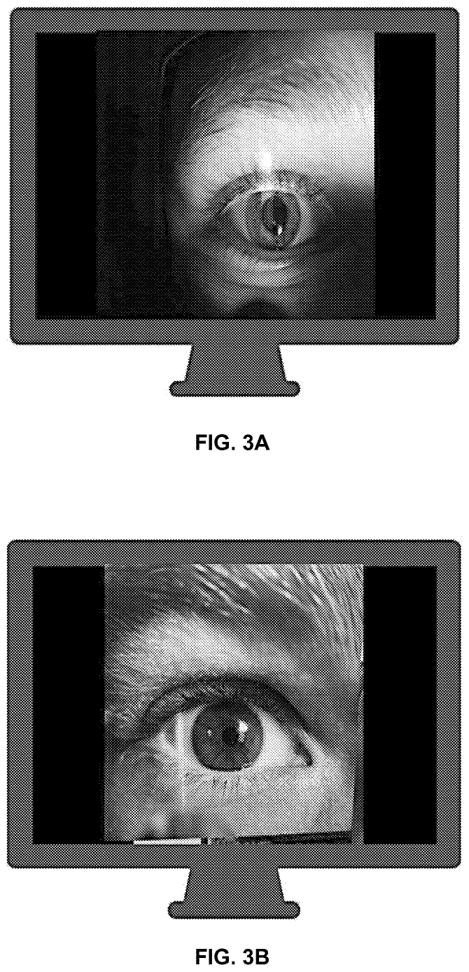

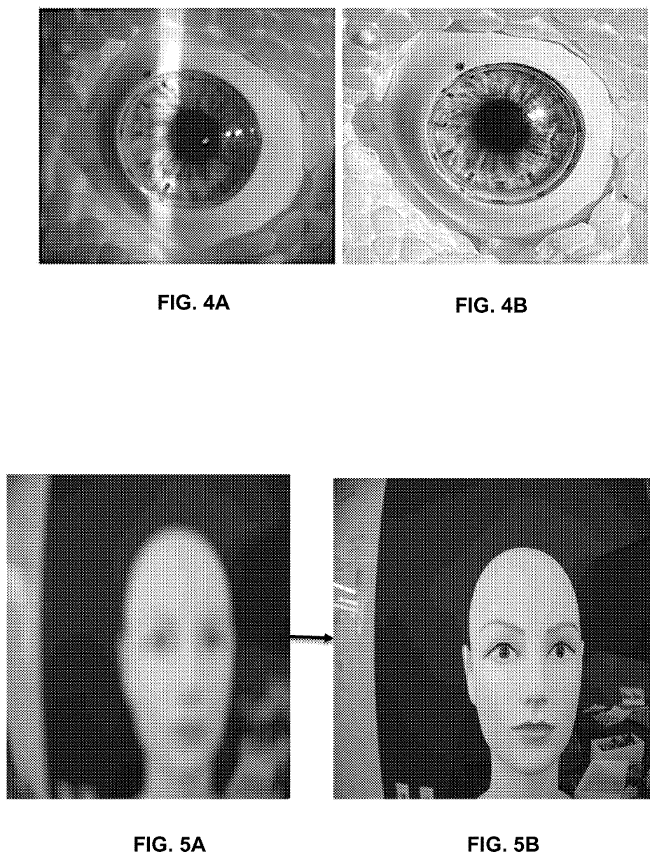

FIGS. 3A-3B are images of pairs of brown eyes and blue eyes, respectively, captured from the slit lamp. Videos were taken to showcase the movement of the slits (FIGS. 4A-4B). Overall the superior clarity and focus of the slit on the eye (FIG. 5A) is compared to an image of the eye taken with smart phone (FIG. 5B). It is contemplated that fine-tuning the brightness of the ambient light, as well as of the slit LEDs, may ensure clarity of the scans.

### Example 3

### Integration of a Dual-Camera Stereoscopic Imaging System

A stereoscopic imaging prototype was assembled using dual IMX477 camera modules mounted on a custom adjustable rig to simulate the headset configuration (FIGS. 1C, 1D). The cameras were positioned with a horizontal baseline offset to create a stereoscopic setup in which the two views differed primarily by lateral translation along a single axis. Preliminary convergent alignment configurations, in which the optical axes were angled slightly inward toward the target, were also tested. Recordings of a model eye and mannequin were obtained across setups with different focal lengths of 10 and baseline separations at fixed working distances of 10-15 cm. The resulting side-by-side video pairs were reviewed through a VR display headset (Meta Quest 3S) to identify configurations that provided optimal image clarity, depth perception, and viewer comfort.

### Validation of the Imaging System

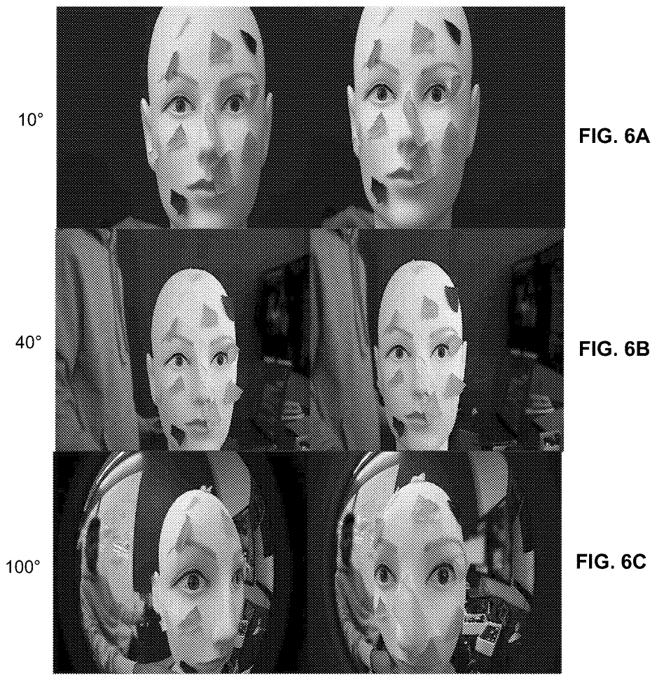

Stereoscopic validation demonstrated that the dual-camera setup reliably reproduced depth perception. Longer focal lengths produced superior image detail and stereoscopic comfort, while baseline variation had a limited impact within the tested range (~4-7 cm). Preliminary convergent alignment improved target centering by bringing the eye more consistently into the shared field of view. This demonstrates the feasibility of a headset-based slit lamp that combines the portability required for telemedicine with high-quality, depth-resolved imaging. Representative stereoscopic image pairs from mannequin-eye testing at about 10°, about 40°, and about 100° fields of view (FOV) (FIGS. 6A-6C). Lenses with narrower FOV caused less observer eye strain and improved stereopsis. Inter-camera spacing did not affect observer depth perception, comfort, or clarity.

What is claimed is:

1. A portable device for an ophthalmic examination, comprising:
a headset comprising a slit lamp integrated therein with a head strap attached thereto;
means for remote access to results of the ophthalmic examination; and
a power source.

2. The portable device of claim 1; wherein the head strap is adjustable.

3. The portable device of claim 1, wherein the slit lamp comprises:
a motorized light system with a slit;
at least one camera module; and
means for ambient lighting.

4. The portable device of claim 3, further comprising:
means for X-Y-Z movement of the motorized light system;
an autofocus; or
stereoscopic imaging; or
a combination thereof.

5. The portable device of claim 3, wherein the motorized light system with a slit comprises a pair of slit LEDs.

6. The portable device of claim 3, wherein the at least one camera module comprises at least one camera for still images and at least one camera for live video imaging.

7. The portable device of claim 3, wherein the at least one camera module is a dual-camera stereoscopic imaging assembly.

8. The portable device of claim 3, wherein the ambient lighting means comprises an ambient LED.

9. The portable device of claim 1, wherein the remote access means comprises a microcontroller and at least one network connection.

10. The portable device of claim 1, wherein the portable device is configured to examine anterior segments of both eyes.

11. The portable device of claim 10, wherein the anterior segments are segments of a cornea, of an iris or of a lens or a combination thereof.

12. A method for performing a vision examination on a patient in need thereof, comprising:
positioning the portable device of claim 1 over the patient's head such that at least one camera in the slit lamp align with at least one eye,
obtaining images of at least one eye via the at least one camera during the vision examination; and
transmitting wirelessly the images to a healthcare provider.

13. The method of claim 12, wherein the obtaining step comprises obtaining images of both eyes via cameras in a dual-camera stereoscopic imaging assembly.

14. The method of claim 12, wherein the patient is in a location remote from the healthcare provider; wherein the transmitting step comprises transmitting wirelessly the images during a telemedicine consultation.

15. A portable slit lamp configured for examination of anterior structures of both eyes, comprising:
a headset comprising:
at least one internal motor;
a slit motorized via one of the internal motors;
a modifiable light source aligned with the slit;
means for capturing images of the eye;
a microcontroller with at least one network connection; and
a power supply.

16. The portable slit lamp of claim 9, wherein the headset further comprises at least one of:
means for moving the modifiable light source in X-Y-Z directions;
an autofocus; or
stereoscopic imaging.

17. The portable device of claim 15, wherein the modifiable light source comprises a pair of slit LEDs and an ambient LED.

18. The portable device of claim 15, wherein the capturing means comprises at least one camera for still images and at least one camera for live video.

19. The portable device of claim 15, wherein the capturing means comprises a dual-camera stereoscopic imaging assembly.

20. The portable device of claim 15, wherein the anterior structures of the eye are a cornea, an iris and a lens.
